(19) United States
(12) Patent Application Publication
(10) Pub. No.: US 2019/0343152 A1
(43) Pub. Date: Nov. 14, 2019

(54) KOMBUCHA ALCOHOL AND METHOD

(71) Applicants: Deborah Kuhl, Waterloo (CA); Keith Phillip Bridge, Waterloo (CA)

(72) Inventors: Deborah Kuhl, Waterloo (CA); Keith Phillip Bridge, Waterloo (CA)

(21) Appl. No.: 15/976,787

(22) Filed: May 10, 2018

(51) Int. Cl. A23L 2/38; A23F 3/16; A23F 3/18; C12G 3/02; C12N 1/18; C12N 1/20

(52) U.S. Cl. CPC A23L 2/382; A23F 3/166; A23F 3/18; A23V 2002/00; C12N 1/18; C12N 1/20; C12G 3/025

(57) ABSTRACT

A method for making kombucha alcohol; the method includes a preparing a symbiotic culture, fermenting a sweetened tea with the symbiotic culture to provide a fermented tea drink, and combining an alcohol beverage to the fermented tea drink. The method for making kombucha alcohol is useful for combining the properties associated with alcohol and Kombucha for human consumption.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of beverages of existing art and more specifically relates to a fermented tea beverage.

### RELATED ART

Kombucha, also known as tea mushroom or Medusomyces gisevii, is a variety of fermented tea drinks commonly intended as functional beverages for their health benefits. Kombucha is produced by fermenting tea using a symbiotic colony of several species of bacteria and yeasts with complex metabolic pathways. The composition of the colony varies from each fermentation batch. During the fermentation process, Kombucha naturally develops an alcohol content. The alcohol content of the kombucha is usually less than 1%.

Beneficial and therapeutic effects have been attributed to the consumption of Kombucha. Benefits may include but not limited to positive effects in treating asthma, cataracts, diabetes, diarrhea, gout, herpes, insomnia, and rheumatism. In recent years, Kombucha started to become packaged and sold commercially. Slight variations have been made to the preparation of Kombucha (i.e. flavor, process, etc.). However, the utilization of alcoholic beverages combined with Kombucha to create unique flavoring profiles with natural benefits have yet to be explored. Therefore, a suitable solution is desired.

U.S. Pat. No. 9,532,589 to Mariya Andreevna Skripitsyna relates to a consortia and strains of microorganisms, and methods of use thereof. The described consortia and strains of microorganisms, and methods of use thereof relates to biotechnology, food industry and concerns to microbial consortia and microbial and yeast strains, as well as to methods for producing by integrated technological cycle with the use of consortia and microbial and yeast strains from the fermented base, which is a semi-product of bread kvass, fermented kvass, nonalcoholic kvass, as well as to methods for producing tea fungus culture fluid, tea-fungus concentrates, kombucha beverages, and vegetable extracts in a single technology process.

### SUMMARY OF THE INVENTION

In view of the foregoing disadvantages inherent in the known fermented tea beverage art, the present disclosure provides a novel kombucha alcohol and method. The general purpose of the present disclosure, which will be described subsequently in greater detail, is to provide an efficient and effective method for making kombucha alcohol.

A method for making kombucha alcohol is disclosed herein. The method includes preparing a symbiotic culture. The symbiotic culture includes a yeast component and a bacteria component. The method may further include fermenting a sweetened tea with the symbiotic culture to provide a fermented tea drink and combining an alcohol beverage to the fermented tea drink. The alcohol beverage and the fermented tea drink is suitably mixed to provide kombucha alcohol. Moreover, kombucha alcohol comprises in formulaic combination the alcohol beverage and the fermented tea drink.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, kombucha alcohol and method, constructed and operative according to the teachings of the present disclosure.

FIG. 1 flow diagram illustrating a method of making kombucha alcohol, according to an embodiment of the present disclosure.

FIG. 2 is a data table comprising the ingredients of the kombucha alcohol of FIG. 1, according to an embodiment of the present disclosure.

FIG. 3 is a data table comprising the ingredients of a fermented tea drink of FIG. 1, according to an embodiment of the present disclosure.

FIG. 4 is a perspective view of the fermented tea drink of FIG. 1, according to an embodiment of the present disclosure.

FIG. 5 is a perspective view of the kombucha alcohol during an ‘in-use’ condition, according to an embodiment of the disclosure.

The various embodiments of the present invention will hereinafter be described in conjunction with the appended drawings, wherein like designations denote like elements.

### DETAILED DESCRIPTION

As discussed above, embodiments of the present disclosure relate to a fermented tea beverage and more particularly to a kombucha alcohol and method as used to improve the method of combining an alcohol beverage with the benefits and flavoring profile of Kombucha for beneficial results.

Generally, Kombucha and alcoholic beverages are packaged and commercially sold separately. The present disclosure may create a method for preparing unique recipes when combining a fermented tea drink (i.e. Kombucha) with an alcohol beverage (i.e. wine, spirits, etc.). The method may result in an admixture that promotes the healthy benefits of Kombucha and the effects of alcohol. The present disclose may provide a low-calorie and healthier alternative to current recipes for alcoholic beverages.

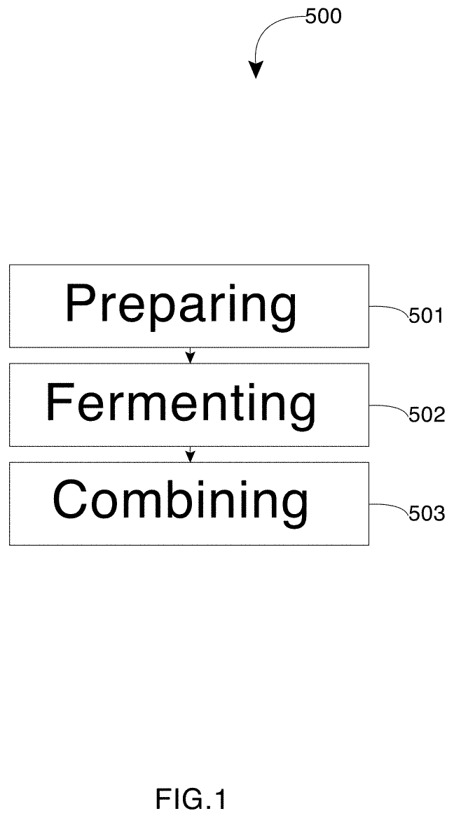

Referring now more specifically to the drawings by numerals of reference, there is shown in FIGS. 1-5 of a kombucha alcohol 100.

FIG. 1 is a flow diagram illustrating a method for making kombucha alcohol 500, according to an embodiment of the present disclosure. In particular, the method for making kombucha alcohol 500 may include one or more components or features of the kombucha alcohol 100. As illustrated, the method for making kombucha alcohol 500 may include the steps of: step one 501, preparing a symbiotic culture 110, the symbiotic culture 110 including a yeast component and a bacteria component; step two 502, fermenting a sweetened tea 112 with the symbiotic culture 110 to provide a fermented tea drink 120; and step three 503, combining an alcohol beverage 130 to the fermented tea drink 120. The alcohol beverage 130 and the fermented tea drink 120 may be suitably mixed to provide the kombucha alcohol 100. Those with ordinary skill in the art will now appreciate that upon reading this specification and by their understanding the art of fermenting as described herein, methods of making kombucha will be understood by those knowledgeable in such art.

It should be noted that the steps described in the method 500 can be carried out in many ways known in the art according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for fermenting the fermented tea drink 120 (e.g., different step orders within above-mentioned list, elimination or addition of certain steps, including or excluding certain maintenance steps, etc.), are taught herein.

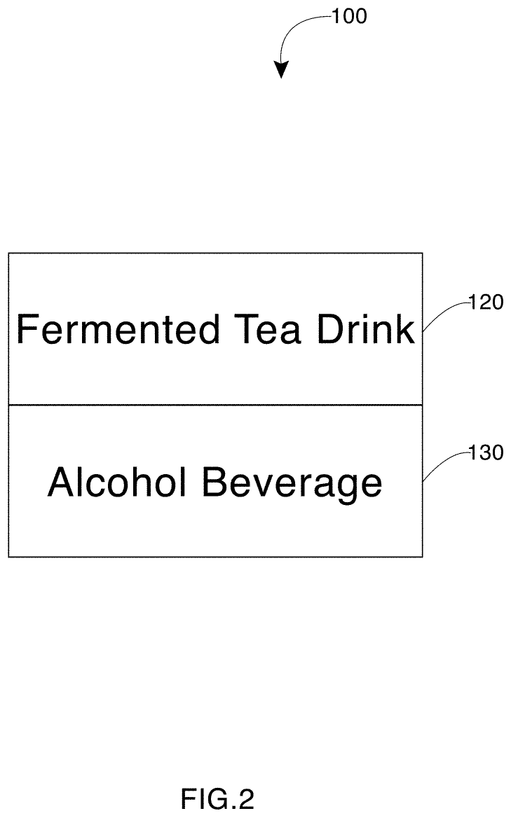

Referring now to FIG. 2 showing a data table of the ingredients in the kombucha alcohol 100 of FIG. 1, according to an embodiment of the present disclosure. As above, the kombucha alcohol 100 may include in formulaic combination the alcohol beverage 130 and the fermented tea drink 120. The alcohol beverage 130 and the fermented tea drink 120 may be suitably mixed to provide the kombucha alcohol 100. The kombucha alcohol 100 may comprise an admixture for promoting a healthy beverage containing an alcohol content of at least 1% of total weighted volume.

One example according to the present embodiment, the alcohol beverage 130 includes a fermented grape juice (i.e. wine). The alcohol beverage 130 may be distilled from a fermented alcohol prior to combining with the fermented tea drink 120, such that the original fermentation benefits are not harmed in both beverages. In another embodiment, the alcohol beverage 130 may be selected from a group consisting of spirits, liquor, and yeast-fermented malt (and the like).

A variety of combinations and ratios may be utilized between the fermented tea drink 120 and the alcohol beverage 130. One example within the scope of the kombucha alcohol 100 may include 2:1 admixture of the fermented tea drink 120 to the fermented grape juice. In another example, the kombucha alcohol 100 may include a 1:2 admixture of the fermented tea drink 120 to the fermented grape juice.

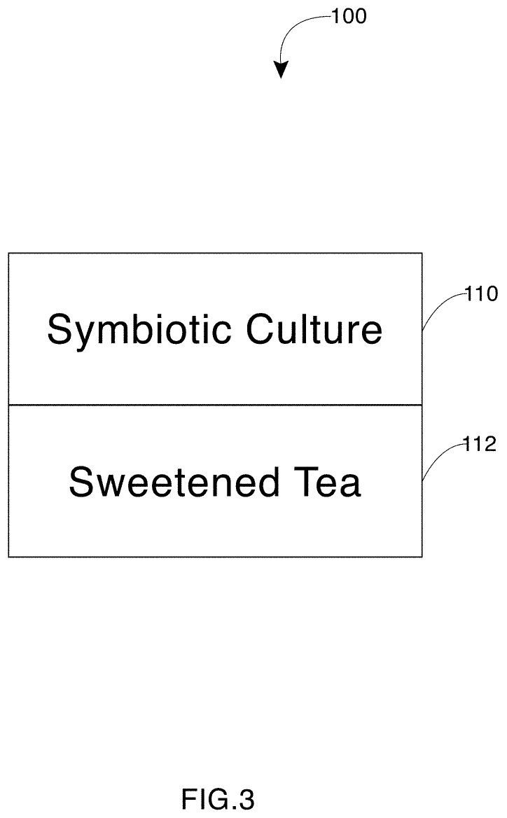

FIG. 3 is a data table comprising the ingredients of the fermented tea drink 120 of FIG. 1, according to an embodiment of the present disclosure. The fermented tea drink 120 (i.e. Kombucha) may comprise of the symbiotic culture 110 and the sweetened tea 112. The symbiotic culture 110 includes the yeast component and the bacteria component. The bacteria component may include Gluconacetobacter xyylinus as one strain identified within the symbiotic culture 110. The bacteria component may oxidize to provide a byproduct of various acids. The various acids may include gluconic acid and acetic acid (among others) for providing antiseptic and antibacterial properties.

The yeast component may include at least one fungi species, the at least one fungi species including Saccharomyces cerevisiae. Subspecies of Saccharomyces cerevisiae may also be found in the symbiotic culture 110. For example, Saccharomyces Boulardii may be identified and provide probiotic benefits and improved digestion for the user. The at least one fungi species may be developed naturally from the original fermentation process of the symbiotic culture 110. The symbiotic culture 110 may be prepared by combining raw-kombucha with the sweetened tea 112 and allowing a predetermined number of days (i.e. 10-14 days) for fermentation. Once the symbiotic culture 110 is prepared, the symbiotic culture 110 may be fermented with a different batch of the sweetened tea 112 to provide the fermented tea drink 120.

The sweetened tea 112 may be prepared from a tea leaf soaked in a hot water bath and a dissolved sweetener. The dissolved sweetener may be selected from a group of natural sweeteners consisting of cane sugar, syrup, and agave. Further, the tea leaf may be selected from a black tea leaf or a green tea leaf. However, additional tea leaves may be contemplated within the scope of the present disclosure.

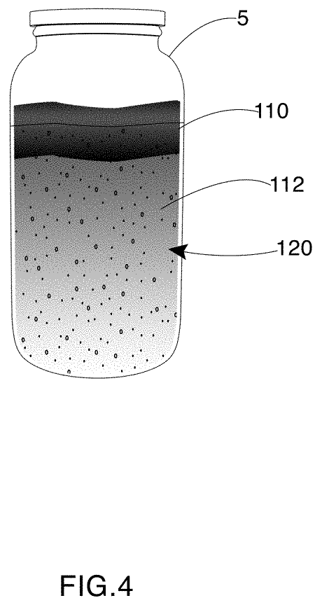

Referring now to FIG. 4, a perspective view of the fermented tea drink 120 of FIG. 1, according to an embodiment of the present disclosure. The fermented tea drink 120 may be fermented within a sealed container 5. The sealed container 5 may be sealed with a lid comprised of a woven cloth and a rubber band or other methods known in the art. Fermenting 502 may be approximately 6-10 days long or as desired. Additionally, fermenting 502 may take place at room temperature, the room temperature being 68-77 degrees Fahrenheit.

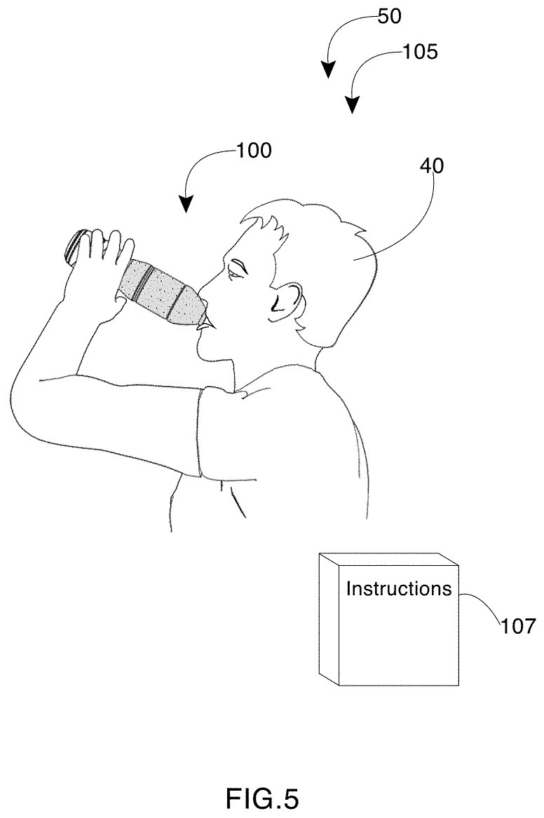

FIG. 5 shows the kombucha alcohol 100 during an ‘in-use’ condition 50, according to an embodiment of the present disclosure. Here, the kombucha alcohol 100 may be beneficial for use by a user 40 to provide properties associated with alcohol and Kombucha. As illustrated, the kombucha alcohol 100 may include the fermented tea drink 120 and the alcohol beverage 130. The alcohol beverage 130 and the fermented tea drink 120 may be suitably mixed to provide the kombucha alcohol 100. The kombucha alcohol 100 comprises in formulaic combination the alcohol beverage 130 and the fermented tea drink 120. The kombucha alcohol 100 may be packaged in bottles or containers for human consumption.

According to one embodiment, the kombucha alcohol 100 may be arranged as a kit 105. In particular, the method for making the kombucha alcohol 500 may further include a set of instructions 107. The instructions 107 may detail preparation of the kombucha alcohol 100 such that the kombucha alcohol 100 can be used, maintained, packaged, prepared, or the like in a preferred manner.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A method for making kombucha alcohol comprising:
preparing a symbiotic culture, said symbiotic culture including,
a yeast component, and
a bacteria component;
fermenting a sweetened tea with said symbiotic culture to provide a fermented tea drink;
combining an alcohol beverage to said fermented tea drink;
wherein said alcohol beverage and said fermented tea drink is suitably mixed to provide kombucha alcohol; and
wherein said kombucha alcohol comprises in formulaic combination said alcohol beverage and said fermented tea drink.

2. The method of claim 1, wherein said kombucha alcohol comprises an admixture for promoting a healthy beverage containing an alcohol content of at least 1% of total weighted volume.

3. The method of claim 1, wherein said yeast component includes at least one fungi species.

4. The method of claim 3, wherein said at least one fungi species includes Saccharomyces cerevisiae.

5. The method of claim 1, wherein said bacteria component includes Gluconacetobacter xyylinus.

6. The method of claim 1, wherein said sweetened tea is made from a tea leaf soaked in a hot water bath and a dissolved sweetener.

7. The method of claim 6, wherein said tea leaf includes a black tea leaf.

8. The method of claim 6, wherein said tea leaf includes a green tea leaf.

9. The method of claim 6, wherein said dissolved sweetener is selected from a group of natural sweeteners consisting of cane sugar, syrup, and agave.

10. The method of claim 1, wherein said fermenting takes place at room temperature, said room temperature being 68-77 degrees Fahrenheit.

11. The method of claim 1, wherein said fermenting is approximately 6-10 days.

12. The method of claim 1, wherein said fermenting is produced within a sealed container.

13. The method of claim 1, wherein said alcohol beverage is distilled from a fermented alcohol prior to combining with said fermented tea drink.

14. The method of claim 1, wherein said alcohol beverage includes a fermented grape juice.

15. The method of claim 1, wherein said bacteria component oxidizes to provide a byproduct of various acids.

16. The method of claim 15, wherein said various acids include gluconic acid and acetic acid.

17. A method for making kombucha alcohol comprising:
preparing a symbiotic culture, said symbiotic culture including,
a yeast component, and
a bacteria component;
fermenting a sweetened tea with said symbiotic culture to provide a fermented tea drink;
combining an alcohol beverage to said fermented tea drink;
wherein said alcohol beverage and said fermented tea drink is suitably mixed to provide kombucha alcohol;
wherein said kombucha alcohol comprises in formulaic combination said alcohol beverage and said fermented tea drink;
wherein said kombucha alcohol comprises an admixture for promoting a healthy beverage containing an alcohol content of at least 1% of total weighted volume;
wherein said yeast component includes at least one fungi species;
wherein said at least one fungi species includes Saccharomyces cerevisiae;
wherein said bacteria component includes Gluconacetobacter xyylinus;
wherein said sweetened tea is made from a tea leaf soaked in a hot water bath and a dissolved sweetener;
wherein said tea leaf includes a black tea leaf;
wherein said fermenting takes place at room temperature, said room temperature being 68-77 degrees Fahrenheit;
wherein said fermenting is approximately 6-10 days;
wherein said fermenting is produced within a sealed container;
wherein said alcohol beverage is distilled from a fermented alcohol prior to combining with said fermented tea drink;
wherein said alcohol beverage includes a fermented grape juice;
wherein said bacteria component oxidizes to provide a byproduct of various acids; and
wherein said various acids include gluconic acid and acetic acid.

18. The method of making kombucha alcohol of claim 17, further comprising set of instructions; and
wherein the kombucha alcohol is arranged as a kit wherein its components are packaged separately.
